(19) United States
(12) Patent Application Publication
(10) Pub. No.: US 2005/0129640 A1
(43) Pub. Date: Jun. 16, 2005

(54) Liquid cosmetic composition comprising gellan gum or a derivative thereof, a solid compound, and a monovalent salt, processes for using this composition, and methods of using same

(75) Inventor: Ludivine Laurent, Courbevoie (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(73) Assignee: L'OREAL

(21) Appl. No.: 10/990,517

(22) Filed: Nov. 18, 2004

Related U.S. Application Data
(60) Provisional application No. 60/562,257, filed on Apr. 15, 2004.

(30) Foreign Application Priority Data
Nov. 18, 2003 (FR) 0313488

(51) Int. Cl. A61K 7/021; A61K 7/06; A61K 7/11

(52) U.S. Cl. 424/63; 424/70.13

(57) ABSTRACT

Disclosed herein is a non-washing aqueous liquid cosmetic composition comprising, in a cosmetically acceptable medium, at least one gellan gum, or a derivative thereof, at least one solid compound, and at least one monovalent salt. Also disclosed is a process for shaping or holding the hairstyle, using the cosmetic composition. Also disclosed are cosmetic products, such as hair care compositions, hair conditioning compositions, and make-up compositions for the hair or the integuments.

This application claims benefit of U.S. Provisional Application No. 60/562,257, filed Apr. 15, 2004, which is incorporated by reference herein in its entirety.

The present disclosure relates to non-washing aqueous liquid cosmetic compositions comprising, in a cosmetically acceptable medium, at least one gellan gum or a derivative thereof, at least one solid compound, and at least one monovalent salt. The disclosure also relates to a process for shaping or holding a hairstyle in which these cosmetic compositions are used. Additionally, this disclosure relates to methods of using these compositions for example, shaping or holding a hairstyle.

It is common practice in the cosmetics field to use compositions comprising solid particles. Recently, there has been a trend towards adding iridescent or nacreous compounds to cosmetic compositions, and this trend has resulted in a resurgence of, presence, and use of these compositions in the cosmetic market. Thus, compositions comprising solid particles in suspension have appeared in makeup products for the skin and the integuments, such as eyelashes, nails, and other keratinous materials, body washing compositions, shampoos and hair shaping compositions.

One problem posed by these compositions is their lack of stability. The stability of compositions containing suspensions of solid particles is first linked to the viscosity of the support composition, and second to the maintenance of the homogeneity of the distribution of the solid particles in the support composition. Following a rise in temperature or simply over time, it is common to find sedimentation of the solid particles (when the density of the solid particles is greater than the density of the support) or creaming (when the density of the solid particles is less than the density of the support) in these compositions.

All kinds of gelling agents have been used in an attempt to obtain stable compositions comprising solid particles in suspension, including polymers or copolymers of unsaturated organic carboxylic acids or of unsaturated esters, polysaccharide derivatives, gums, colloidal silicates, polyethylene glycols, polyvinylpyrrolidones, and hydrophilic silica gels. However, the results obtained with these gelling agents are not always satisfactory and may vary with the nature of the solid particles used.

In patent application FR 2,700,952, filed by L'Oreal, it has been proposed that gels optionally comprising solid particles in suspension can be stabilized with expanded hollow microspheres made of thermoplastic material. The described microspheres, which have a specific mass of from 15 to 200 kg/m³, are added to the compositions in a proportion of from 0.1% to 10% by weight (of microspheres) relative to the total weight of the compositions. Additionally, the compositions described in FR 2,700,952 are free of fatty substances.

Patent application WO 02/072063, filed by the company Beiersdorf A.G., describes washing compositions comprising particles in suspension in a mixture of surfactants, xanthan gum, and gellan gum.

The present inventors have discovered, surprisingly and advantageously, for example, that the use of a combination of a gellan gum, or a derivative thereof, and of a monovalent salt makes it possible to stabilize non-washing aqueous liquid compositions containing solid particles.

As used herein, the terms “solid particles” and “solid compounds” are equivalent and used interchangeably; they both refer to compounds or particles that are always in solid form in the final cosmetic compositions.

The combination of a gellan gum, or a derivative thereof, and of a monovalent salt allows uniform placement in suspension of solid particles, even in a medium of very low viscosity (of about from 1 to 10 poises, i.e., 0.1 to 1 Pa.s). And in contrast to the compositions previously used, the cosmetic compositions according to the present disclosure are stable for several months, even when subjected to variations in temperature.

Furthermore, the thixotropic nature of compositions comprising this combination allows for desired restitution of the properties of these styling cosmetic compositions when they are applied by means of a spray or as an aerosol.

The compositions disclosed herein may be used in non-washing aqueous liquid cosmetic compositions. For example, in one embodiment, they may be used in hair shaping or holding compositions.

Another advantage of the disclosed compositions is that they allow for the production of cosmetic compositions that give the hair desirable cosmetic properties, especially in terms of feel (soft feel) and of ease of disentangling.

When the compositions disclosed herein are used in cosmetic compositions for shaping and/or holding the hairstyle, the final cosmetic compositions impart good fixing and good holding qualities to the hair, including a styling effect that persists throughout the day, or even for several days, which resists the effects of water on hair style and hold and is easy to remove by shampooing.

Thus, the present disclosure also relates to a process for shaping or holding a hairstyle involving use of a composition comprising a gellan gum, or a derivative thereof, and a monovalent salt. Moreover, this disclosure relates to the method of using a composition comprising a gellan gum, or a derivative thereof, and a monovalent salt in styling and holding of hair. In one embodiment, the compositions do not contain any standard fixing agents.

Therefore, one aspect of the present disclosure relates to a non-washing aqueous liquid cosmetic compositions containing, in a cosmetically acceptable medium, at least one gellan gum, or a derivative thereof, at least one solid compound, and at least one monovalent salt. In another embodiment, the cosmetic composition disclosed herein is a hair composition, such as a cosmetic hair composition for hair styling. Moreover, the cosmetic hair composition according to the present disclosure may be in the form of a spray, a mousse, or a gel.

Another aspect of the present disclosure includes a process for shaping or holding a hairstyle, using a cosmetic composition as disclosed herein.

Still another aspect of the disclosure concerns a method of using a cosmetic composition as a styling composition for fixing and holding the hair. In one embodiment, the composition is a hair care composition that imparts softness to the hair or a makeup composition for the hair or the integuments.

Thus, the present disclosure relates generally to the use of the disclosed cosmetic composition for fixing hair, providing holding properties to the hair, and giving the hair cosmetic qualities.

Other subjects, characteristics, aspects, and advantages of the disclosure will emerge even more clearly on reading the description and the examples that follow.

In one embodiment, the cosmetic compositions according to the present disclosure may be in the form of a gel, which is known to be a three-dimensional network of molecules that holds in its mesh a large amount of solvent. The formation of such a network is commonly referred to a the process of gelation. The cosmetic compositions may also be in the form of a mousse.

For the purposes of the present disclosure, the term “styling cosmetic composition” means a composition for shaping or holding the hairstyle. In addition, the term “liquid composition” means that the viscosity of the composition ranges from the viscosity of water to 50 poises, such as ranging from the viscosity of water to 20 poises.

As used herein, the term “aqueous composition” means that the cosmetically acceptable medium used in the compositions according to the present disclosure is an aqueous medium optionally containing at least one organic solvent. The at least one organic solvent that may be used in the compositions of the present disclosure may be chosen from C₁-C₄ lower alcohols, for instance ethanol, isopropanol, tert-butanol or n-butanol, polyols, for instance propylene glycol, polyol ethers, and mixtures thereof. In one embodiment, the alcohol is ethanol.

The aqueous compositions of the present disclosure may contain less than about 10% oily compounds (by weight); the term “oily compounds,” as it is used in this disclosure, includes both liquid and solid oily compounds.

The compositions according to the disclosure are “non-washing cosmetic compositions,” meaning that the compositions do not contain more than 4% of surfactants by weight relative to the total weight of the composition.

Gellan gum is a polysaccharide produced by aerobic fermentation of Sphingomonas elodea, more commonly known as Pseudomonas elodea. This linear polysaccharide contains sequences of the following monosaccharides: D-glucose, D-glucuronic acid, and L-rhamnose. In its native state, gellan gum is highly acylated.

The gellan gum used in various embodiments of the compositions according to the present disclosure is at least partially deacylated gellan gum. This at least partially deacylated gellan gum is obtained by a high-temperature alkaline treatment, such as in a KOH or NaOH solution. For example, the purified gellan gum sold under the trade name Kelcogel® by the company Kelco is suitable for preparing the compositions according to the disclosure.

Gellan gum derivatives may be obtained by performing standard chemical reactions such as esterifications or addition of a salt of an organic or mineral acid. An example of a gellan gum derivative that may be used in the present disclosure is welan gum. Welan gum is a gellan gum modified by fermentation using the Alcaligenes strain ATCC 31 555. Welan gum has a repeating pentasaccharide structure formed from a main chain consisting of D-glucose, D-glucuronic acid, and L-rhamnose units on which is grafted a pendent L-rhamnose or L-mannose unit. The welan gum sold under the trade name Kelco Crete® by the company Kelco is suitable for preparing the compositions disclosed herein.

The concentration of gellan gum, or derivatives thereof, used in the compositions disclosed herein ranges from 0.005% to 10% by weight relative to the total weight of the composition. In one embodiment, the concentration of gellan gum, or derivatives thereof, range from 0.01% to 5%, such as from 0.02% to 3% by weight relative to the total weight of the composition.

The monovalent salts that may be used in the disclosed compositions are salts of monovalent cations such as alkali metal salts, ammonium salts, organic amine salts, or mixtures thereof. The monovalent cations of the alkali metals are the following cations: Li⁺, Na⁺, K⁺, Rb⁺, Cs⁺, Fr⁺. In one embodiment, Na⁺ is used in the compositions according to the present disclosure. The counterion may be a mineral or organic anion, such as Cl⁻, and the salt may be NaCl.

The concentration of monovalent salt(s) used herein ranges from 0.01% to 10% by weight relative to the total weight of the composition, such as from 0.05% to 5% by weight relative to the total weight of the composition.

In one embodiment, the ratio of monovalent salt/gellan gum or its derivative ranges from 1% to 50%, such as from 2% to 30%.

The solid compounds or solid particles that may be used in the disclosed cosmetic compositions are cosmetically acceptable compounds that are solid under conditions of room temperature (between 20° C. and 40° C.) and standard atmospheric pressure. Any organic, mineral, or hybrid solid, of any form, having a solubility in the cosmetically acceptable medium of less than about 0.01 g/100 g may be used. As an example, hollow spheres that meet the required solubility range may be used. In one embodiment, the solid compounds are non-polymeric solid compounds, and are not in the form of capsules containing an active principle.

Among the solid compounds that may be used are solid nacreous agents or opacifiers, solid pigments, solid sunscreens, insoluble non-lamellar pulverulent compounds, agar spheres, polysaccharide spheres, and active particles, such as selenium sulfide, zinc pyrithione, waxes such as apple wax, sunflower wax, candelilla wax, beeswax, carnauba wax, animal or synthetic waxes, and fillers.

The agents that may be used herein are nacreous agents or opacifiers chosen from uncoated titanium oxides, coated titanium oxides, titanium micas, and micas.

The titanium oxides that may be used in the disclosed compositions generally have a particle size ranging from 2 to 500 nanometers (nm), such as from 2 to 300 nm, and 2 to 50 nm.

The uncoated titanium oxides that may be used herein include:
(1) uncoated titanium oxides in powder form:
   a) Bayertitan and Titanium Dioxide A sold by the company Bayer, and
   b) 70110 Cardre UF TiO₂ sold by the company Cardre,
(2) uncoated titanium oxides as an aqueous dispersion containing 10%, 20%, or 30% by weight of titanium oxide relative to the total weight of the aqueous dispersion, and with a particle size of 15, 20, or 60 nm:
   a) Sunveil 1010, Sunveil 1020, Sunveil 1030, Sunveil 2020, Sunveil 2030, Sunveil 6010, and Sunveil 6030 sold by the company Catalysts & Chemicals, and
   b) Micro Titanium Dioxide-USP Grade sold by the company Color Techniques.

The coated titanium oxides that may be used herein include:
(1) polydimethylsiloxane-coated titanium oxides (e.g., Cardre Ultrafine Titanium Oxide as sold by the company Cardre),
(2) polymethylhydrogenosiloxane-coated titanium oxides (e.g., polymethylhydrogenosiloxane-coated untreated titanium oxide sold under the trade name Cosmetic White SA-C47-051-10 by the company Miyoshi),
(3) perfluoropolymethylisopropylether-coated titanium oxides (e.g., Cardre Mica FHC 70173 or 70170 Cardre UF TiO₂ FHC sold by the company Cardre),
(4) silica-coated titanium oxides (e.g., Spherititan AB sold by the company Catalysts & Chemicals),
(5) Teflon-coated titanium oxides (e.g., CS-13997 Teflon Coated Titanium Dioxide sold by the company Clark Colors),
(6) polyester-coated titanium oxides (e.g., Experimental Desoto Beads sold by the company Desoto),
(7) chitosan-coated titanium oxides (e.g., CT-2 Titanium Dioxide MT-500SA sold by the company Dainihon Kasei), and
(8) N-lauryl-L-lysine-coated titanium oxides (e.g., LL-5 Titanium Dioxide A 100, LL-3 Titanium Dioxide MT-100SA, LL-5 Titanium Dioxide CR-50, LL-5 Titanium Dioxide MT-100SA, or LL-5 Titanium Dioxide MT-500SA sold by the company Dainihon Kasei).

It is also possible to use white nacreous pigments, such as mica coated with titanium oxide or with bismuth oxychloride, colored nacreous pigments, such as titanium mica colored with iron oxides or with chromium oxide, titanium mica colored with an organic pigment such as the organic pigments described below, or nacreous pigments based on bismuth oxychloride.

The titanium micas that may be used herein include the following products:
(1) Flonac FS 20 C, Flonac ME 10 C, Flonac MG 10 C, Flonac MI 10 C, Flonac ML 10 C, and Flonac MS 10 C sold by the company Eckart,
(2) Timica Iridescent Red or Mattina Green sold by the company Engelhard, and
(3) Timiron Green MP-165 (17212), Timiron Starluster MP-115 (17200), or Timiron Super Sparkle MP-148 (17297) sold by the company Merck.

Non-limiting examples of micas that may be used herein include Cosmetic Mica 280 BC from Whittaker Clark D and Mearlmica MMSV from Engelhard.

In another embodiment, the compositions disclosed herein include micas or titanium micas.

The solid pigments that may be used include mineral pigments such as titanium dioxide, black, yellow, red or brown iron oxides, manganese violet, ultramarine violet, ultramarine blue, chromium oxide, and organic pigments such as the pigments D&C Red No 3, No 6, No 7, No 9, No 13, No 19, No 21, No 27, No 30, No 36, and carbon black.

The agents that may be used in the compositions according to the present disclosure as sunscreen agents are chosen from solid benzylidenecamphors, solid triazines, solid benzophenones, solid dibenzoylmethanes, solid PABAs (para-aminobenzoic acids), solid benzotriazoles, solid cinnamates, and solid imidazolines.

Solid benzylidenecamphors include 3-benzylidenecamphor, 4-methylbenzylidenecamphor, and polyacrylamidomethylbenzylidenecamphor. One example of a solid triazine that may be used is anisotriazine.

The solid benzophenones that may be used in the present disclosure include benzophenone-1, benzophenone-2, benzophenone-3, benzophenone-6, benzophenone-8, benzophenone-9, and benzophenone-12.

Among the solid dibenzoylmethanes are butylmethoxydibenzoylmethane and isopropyldibenzoylmethane and the solid PABAs that may be used in the compositions of the present disclosure include diethylhexylbutamidotriazone, ethyldihydroxypropyl PABA, ethylhexyltriazone, ethyl PABA and glyceryl PABA.

Additionally, the solid benzotriazoles may optionally be chosen from drometrizole trisiloxane and methylenebis(benzotriazolyl)tetramethylbutylphenol.

Finally, the solid cinnamates include etocrylene and isopropyl methoxycinnamate. Ethylhexyldimethoxydibenzylidenedioxoimidazoline propionate is mentioned as a solid imidazoline that may be used in the composition disclosed herein.

Among the insoluble non-lamellar pulverulent compounds that may be used in the disclosed compositions are the following:
(1) talc,
(2) kaolin, which is a hydrated aluminium silicate,
(3) nylon powders (e.g., the product sold under the name Orgasol 2002 ED NAT COS by the company Atochem),
(4) polyethylene powders (e.g., the product sold under the name Coathylene HA 1681 by the company Plast Labor),
(5) poly-β-alanine powders,
(6) polyfluoro powders such as polytetrafluoroethylene powder (e.g., the product sold under the name MP 1400 by the company DuPont de Nemours),
(7) acrylic copolymer powders, such as those sold under the name Polytrap Q5 6603 by the company Dow Chemical,
(8) polystyrene powders, such as those sold under the name Polysphere 3000 SP by the company Presperese,
(9) polyester powders,
(10) expanded thermoplastic microspheres (e.g., the product sold under the name Expancel 551 DE by the company Expancel),
(11) silicone resin microbeads (e.g., Tospearls from the company Toshiba),
(12) zinc oxide and titanium oxide,
(13) aluminium oxide, zirconium oxide, or cerium oxide,
(14) precipitated calcium carbonate, magnesium carbonate, and magnesium hydrocarbonate,
(15) hydroxyapatite,
(16) metal soaps derived from organic carboxylic acids containing from about 8 to 22 carbon atoms or, in some embodiments, from 12 to 18 carbon atoms (e.g., zinc stearate, magnesium stearate, lithium stearate, zinc laurate or magnesium myristate),
(17) silicas,
(18) hydrophilic polymer powders of synthetic origin such as polyacrylates (e.g., the product sold under the name Micropearl M 100 by the company Matsumoto),
(19) acrylic polyamides such as those sold by the company Oris,
(20) insoluble polyurethanes, such as the product sold under the name Plastic Powder D 800 by the company Toshnu, and
(21) porous cellulose microspheres.

The pulverulent compounds may also be compounds that have been made hydrophilic by coating or chemical grafting with products such as chitosan, titanium dioxide, silica or hydrophilic polymers, and especially sulfonic polyesters or polyquaternary ammoniums. For example, the chitosan-coated talc sold by Daito under the name Talc CT 2 MSA may be used as a pulverulent compound.

It is also possible to use hydrophobic pulverulent compounds, chosen from pulverulent compounds of either hydrophobic or hydrophilic nature. Hydrophilic compounds are made hydrophobic by coating or chemical grafting with products such as silicones, amino acids, metal soaps, fluoro derivatives, mineral oils, lecithin, isopropyl triisostearyltitanate, polyethylene, collagen and derivatives thereof, and polyacrylates.

By way of example, polymethylhydrogenosiloxane-coated silica microbeads sold under the trade name Silica SI SB 700 by Miyoshi or the sericite coated with methicone/hydrogenated egg oil, sold under the trade name Sericite SNI S100 by Miyoshi may be used in the present disclosure.

In one embodiment, the size of the pulverulent particles ranges from 10 nm to 100 μm. The “particle size” is the maximum distance that it is possible to measure between two diametrically opposite points on the particle. Particle size may be measured, for example, using a laser granulometer.

The concentration of solid compounds used in the compositions according to the present disclosure may range from 0.001% to 5% by weight relative to the total weight of the composition, such as from 0.01% to 2% by weight relative to the total weight of the composition.

The compositions according to the disclosure may also contain at least one additional adjuvant that is not a solid compound as that term is used here. This additional adjuvant may be chosen from silicones in soluble, dispersed, or microdispersed form, nonionic, anionic, cationic, and amphoteric surfactants, nonionic, anionic, cationic, and amphoteric polymers, ceramides and pseudoceramides, vitamins and provitamins, including panthenol, plant, animal, mineral and synthetic oils, waxes other than ceramides and pseudoceramides, silicone or non-silicone, liquid or solid, water-soluble and liposoluble sunscreens, dyes, sequestering agents, plasticizers, solubilizers, acidifying agents, basifying agents, neutralizers, mineral and organic thickeners, antioxidants, hydroxy acids, glycols, penetrating agents, fragrances, and preserving agents.

A person skilled in the art will be able to select the optional additives and the amount thereof such that they do not harm the desirable properties of the compositions according to the present disclosure.

These additives may be present in the disclosed compositions in an amount ranging from 0 to 20% by weight relative to the total weight of the composition.

The examples that follow illustrate certain embodiments of the present invention and should not in any way be considered as limiting the invention described more broadly in the previous disclosure or the claims.

### EXAMPLES

Compositions formulated as sprays in a pump-dispenser bottle were prepared, in which the contents of the various constituents in g/100 g are as follows:

Composition 1

| Constituent | g/100 g |
|---|---|
| Gellan gum | 0.04 |
| NaCl | 1.0 |
| Agar spheres | 0.15 |
| Fragrance | qs |
| Distilled water | qs 100 |

This composition had a viscosity close to that of water, and the solid particles remained in suspension. After storage for two months at 45° C., no change was observed in the appearance of this composition.

Composition 2

| Constituent | g/100 g |
|---|---|
| Gellan gum | 0.1 |
| NaCl | 0.5 |
| Distilled water | qs 100 |

Application of this composition to the hair gave it an improved styling effect and also a very soft feel.

Composition 3

| Constituent | g/100 g |
|---|---|
| Gellan gum | 0.04 |
| NaCl | 1 |
| Flakes | 0.25 |
| Distilled water | qs 100 |

Composition 4

| Constituent | g/100 g |
|---|---|
| Gellan gum | 0.04 |
| NaCl | 1 |
| Flakes | 0.25 |
| Alcohol | 50 |
| Distilled water | qs 100 |

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

1. A non-washing aqueous liquid cosmetic composition comprising, in a cosmetically acceptable medium (1) at least one gellan gum, or a derivative thereof, (2) at least one solid compound in a concentration ranging from 0.001% to 5% by weight relative to the total weight of the composition, and (3) at least one monovalent salt.

2. The cosmetic composition of claim 1, wherein the gellan gum derivative is a welan gum.

3. The cosmetic composition of claim 1, wherein the concentration of gellan gum or derivatives thereof ranges from 0.005% to 10% by weight relative to the total weight of the composition.

4. The cosmetic composition of claim 3, wherein the concentration of gellan gum or derivatives thereof ranges from 0.01% to 5% by weight relative to the total weight of the composition.

5. The cosmetic composition of claim 4, wherein the concentration of gellan gum or derivatives thereof ranges from 0.02% to 3% by weight relative to the total weight of the composition.

6. The cosmetic composition of claim 1, wherein the solid compound is chosen from organic, mineral, and hybrid solids with a solubility in the cosmetically acceptable medium of less than about 0.01 g/100 g.

7. The cosmetic composition of claim 1, wherein the solid compound is chosen from nacreous agents or opacifiers, solid pigments, solid sunscreens, insoluble non-lamellar pulverulent compounds, agar spheres, polysaccharide spheres, and active particles.

8. The cosmetic composition of claim 7, wherein the solid compound is a nacreous agent or opacifier chosen from uncoated titanium oxides, coated titanium oxides, and micas.

9. The cosmetic composition of claim 8, wherein the micas comprise titanium micas.

10. The cosmetic composition of claim 8, wherein the solid compound is chosen from titanium oxides with a particle size ranging from 2 to 500 nm.

11. The cosmetic composition of claim 10, wherein the particle size ranges from 2 to 50 nm.

12. The cosmetic composition of claim 8, wherein the solid compound is a titanium oxide chosen from uncoated titanium oxides in powder form and uncoated titanium oxides as an aqueous dispersion containing from 10 to 30% of titanium oxide by weight relative to the total weight of the aqueous dispersion and a particle size ranging from 15 to 60 nm.

13. The cosmetic composition of claim 8, wherein the solid compound is chosen from polydimethylsiloxane-coated titanium oxides, polymethylhydrogenosiloxane-coated titanium oxides, perfluoropolymethylisopropylether-coated titanium oxides, silica-coated titanium oxides, Teflon-coated titanium oxides, polyester-coated titanium oxides, chitosan-coated titanium oxides, and N-lauryl-L-lysine-coated titanium oxides.

14. The cosmetic composition of claim 8, wherein the solid compound is chosen from white nacreous pigments, colored nacreous pigments, titanium mica, and nacreous pigments based on bismuth oxychloride.

15. The cosmetic composition of claim 7, wherein the solid compound is a mineral solid pigment chosen from titanium dioxide, black, yellow, red, or brown iron oxides, manganese violet, ultramarine violet, ultramarine blue, and chromium oxide.

16. The cosmetic composition of claim 7, wherein the solid compound is an organic solid pigment chosen from the pigments D&C Red No 3, No 6, No 7, No 9, No 13, No 19, No 21, No 27, No 30, and No 36, and carbon black.

17. The cosmetic composition of claim 7, wherein the solid compound is a solid sunscreen chosen from solid benzylidenecamphors, solid triazines, solid benzophenones, solid dibenzoylmethanes, solid PABAs, solid benzotriazoles, solid cinnamates, and solid imidazolines.

18. The cosmetic composition of claim 7, wherein the solid compound is an insoluble non-lamellar pulverulent compound chosen from talc, kaolin, nylon powders, polyethylene powders, poly-β-alanine powders, polyfluoro powders, acrylic copolymer powders, polystyrene powders, polyester powders, expanded thermoplastic microspheres, silicone resin microbeads, zinc oxide and titanium oxide, aluminium oxide, zirconium oxide or cerium oxide, precipitated calcium carbonate, magnesium carbonate, magnesium hydrocarbonate, hydroxyapatite, metal soaps derived from organic carboxylic acids containing from 8 to 22 carbon atoms, silicas, hydrophilic polymer powders of synthetic origin, acrylic polyamides, insoluble polyurethanes, and porous cellulose microspheres.

19. The cosmetic composition of claim 1, wherein the solid compound is a hydrophilic pulverulent compound having a hydrophilic material coated or chemically grafted thereto.

20. The cosmetic composition of claim 19, wherein the hydrophilic material is chosen from chitosan, titanium dioxide, silica, and hydrophilic polymers.

21. The cosmetic composition of claim 1, wherein the solid compound is a hydrophobic pulverulent compound having a hydrophobic material coated or chemically grafted thereto.

22. The cosmetic composition of claim 21, wherein the hydrophobic material is chosen from silicones, amino acids, metal soaps, fluoro derivatives, mineral oils, lecithin, isopropyl triisostearyltitanate, polyethylene, collagen and derivatives thereof, and polyacrylates.

23. The cosmetic composition of claim 7, wherein the active particles are chosen from selenium sulfide, zinc pyrithione, waxes, and fillers.

24. The cosmetic composition of claim 23, wherein the waxes are chosen from apple wax, sunflower wax, candelilla wax, beeswax, carnauba wax, animal wax, and synthetic wax.

25. The cosmetic composition of claim 14, wherein the white nacreous pigments are chosen from mica coated with titanium oxide or with bismuth oxychloride.

26. The cosmetic composition of claim 14, wherein the colored nacreous pigments are chosen from titanium mica colored with iron oxides or with chromium oxide.

27. The cosmetic composition of claim 14, wherein the titanium mica is colored with an organic pigment.

28. The cosmetic composition of claim 1, wherein the monovalent salt comprises at least one salt of a monovalent cation chosen from alkali metal salt, ammonium salt, and organic amine salt.

29. The cosmetic composition of claim 28, wherein the monovalent alkali metal salt is a Na⁺ containing salt.

30. The cosmetic composition of claim 28, wherein the concentration of monovalent salt ranges from 0.01% to 10% by weight relative to the total weight of the composition.

31. The cosmetic composition of claim 30, wherein the concentration of monovalent salt ranges from 0.05% to 5% by weight relative to the total weight of the composition.

32. The cosmetic composition of claim 1, further comprising at least one additional adjuvant chosen from silicones in soluble, dispersed, or microdispersed form, nonionic, anionic, cationic, and amphoteric surfactants, nonionic, anionic, cationic, and amphoteric polymers, ceramides and pseudoceramides, vitamins and provitamins, waxes other than ceramides and pseudoceramides, silicone or non-silicone, liquid or solid, water-soluble and liposoluble sunscreens, dyes, sequestering agents, plasticizers, solubilizers, acidifying agents, basifying agents, neutralizers, mineral and organic thickeners, antioxidants, hydroxy acids, glycols, penetrating agents, fragrances, and preserving agents.

33. The cosmetic composition of claim 32, wherein the provitamins are chosen from panthenol, plant, animal, mineral, and synthetic oils.

34. The cosmetic composition of claim 32, wherein the at least one additional adjuvant is present in an amount ranging from 0 to 20% by weight relative to the total weight of the composition.

35. The composition of claim 1, wherein the composition is in the form of a gel.

36. The composition of claim 1, wherein the composition is in the form of a mousse.

37. The composition of claim 1, wherein the composition is in the form of a spray.

38. The composition of claim 1, wherein the ratio of monovalent salt to gellan gum or its derivative ranges from 1% to 50%.

39. The composition of claim 1, wherein the concentration of solid compounds ranges from 0.01% to 2% by weight relative to the total weight of the composition.

40. A process for shaping and/or holding a hairstyle comprising applying to hair a non-washing aqueous liquid cosmetic composition comprising, in a cosmetically acceptable medium, (1) at least one gellan gum, or a derivative thereof, (2) at least one solid compound in a concentration from 0.001% to 5% by weight relative to the total weight of the composition, and (3) at least one monovalent salt.

41. The process of claim 40, wherein the cosmetic composition is a styling composition for fixing and holding the hair.

42. The process of claim 40, wherein the cosmetic composition is a haircare composition

43. The process of claim 40, wherein the composition is a hair conditioning composition for giving the hair softness.

44. The process of claim 40, wherein the composition is a makeup composition for the hair or the integuments.

45. The process of claim 44, wherein the integuments are chosen from eyelashes, nails, and other keratinous materials.
